(19) United States
(12) Patent Application Publication
(10) Pub. No.: US 2008/0220520 A1
(43) Pub. Date: Sep. 11, 2008

(54) CRYOPRESERVATION OF HUMAN EMBRYONIC STEM CELLS IN MICROWELLS

(76) Inventors: Sean P. Palecek, Madison, WI (US); Juan J. DePablo, Madison, WI (US); Jeffrey C. Mohr, Madison, WI (US); Lin Ji, Madison, WI (US)

Correspondence Address: QUARLES & BRADY LLP, 33 E. MAIN ST, SUITE 900, P.O. BOX 2113, MADISON, WI, 53701-2113, US

(21) Appl. No.: 12/032,986

(22) Filed: Feb. 18, 2008

Related U.S. Application Data
(63) Continuation-in-part of application No. 10/993,468, filed on Nov. 19, 2004.
Application No. 10/993,468 is a continuation-in-part of application No. 11/765,831, filed on Jun. 20, 2007.
(60) Provisional application No. 60/523,343, filed on Nov. 19, 2003.
(60) Provisional application No. 60/814,975, filed on Jun. 20, 2006.

(51) Int. Cl. C12N 5/06; C12M 1/00

(52) U.S. Cl. 435/374; 435/307.1

(57) ABSTRACT

The present invention relates to methods and structures for preparing stem cells for use in cryopreservation methods. Stem cell colonies are provided between first and second matrix portions and are exposed to a carbohydrate-containing cryoprotecting medium and a freezing medium. The methods of the invention yield cryopreserved cells that maintain cell viability and exhibit limited cell differentiation after freezing and thawing, facilitating storage, shipping and handling of embryonic stem cell stocks and lines for research and therapeutics.

### CROSS-REFERENCE TO RELATED APPLICATIONS

This application is a continuation-in-part of U.S. patent application Ser. No. 10/993,468, filed Nov. 19, 2004, which claims the benefit of U.S. Provisional Patent Application No. 60/523,343, filed Nov. 19, 2003. This application is also a continuation-in-part of U.S. patent application Ser. No. 11/765,831, filed Jun. 20, 2007, which claims the benefit of U.S. Provisional Patent Application No. 60/814,975, filed Jun. 20, 2006. Each application is incorporated herein by reference as if set forth in its entirety.

### STATEMENT REGARDING FEDERALLY SPONSORED RESEARCH OR DEVELOPMENT

This invention was made with United States government support awarded by the following agencies: NAVY/ONR N66001-02-C-8051; NSF-MRSEC 0520527; and NIH HHSN309200582085C. The United States government has certain rights in this invention.

### BACKGROUND

The invention relates generally to methods of preparing embryonic stem cells for cryopreservation, to structures formed in the methods, and to related cryopreservation methods.

Methods for isolating stable undifferentiated hESC cultures were described by Thomson et al, in U.S. Pat. No. 5,843,780 and Thomson J, et al., “Embryonic stem cell lines derived from human blastocysts,” Science 282:1145-1147 (1998), each of which is incorporated herein by reference as if set forth in its entirety.

Recovery of human embryonic stem cells (hESCs) following cryopreservation (i.e., freezing and thawing) is very low. Generally, less than 1% of the hESCs survive, and a significant number of the hESCs that survive undergo differentiation from cryopreservation-induced stresses. Stem cell differentiation can be measured by various well-known methods, for example by monitoring the presence of stem cell surface markers OCT4 and SSEA-4 using immunofluorescence microscopy.

Over the past several years, however, significant progress has been made in cryopreservation and lyophilization of biological systems. Most preservation protocols for living cells rely on the addition of dimethyl sulfoxide (DMSO) at concentrations from 5% to 20%. McLellan M & Day J, “Cryopreservation and freeze-drying protocols. Introduction,” Methods Mol. Biol. 38:1-5 (1995). Other chemicals such as glycerol, ethylene glycol, hydroxycellulose or the disaccharides sucrose, maltose and trehalose have been shown to enhance cell viability when combined with DMSO. Gulliksson H, “Additive solutions for the storage of platelets for transfusion,” Transfus. Med. 10:257-264 (2000). Presumably, these treatments stabilize cell membranes and/or cell proteins during freezing and drying by forming a glassy material at or near the surface of these cell structures.

Typically, mammalian cells are stored in liquid nitrogen for long-term applications, but they can also survive shorter time periods at temperatures near −80°, achievable in low-temperature freezers. As temperatures increase, cellular reaction and oxidative stress rates increase, shortening the time cells remain viable. The goal is to maximize storage temperature, preferably to temperatures of a standard single-compressor freezer (−20° C.), and still maintain a reasonable storage time.

A suitable protectant interacts favorably with cells and other biological materials, is nontoxic, protects during both freezing and drying, substitutes for water and has a high glass transition temperature. Based on recent work by researchers, several disaccharides have been found to satisfy these criteria. In particular, trehalose-based formulations have shown promise. Trehalose is a disaccharide found at high concentrations in a wide variety of organisms that are capable of surviving almost complete dehydration. Crowe J, et al., “Anhydrobiosis,” Annu. Rev. Physiol. 54:579-599 (1992). Trehalose stabilizes certain cells during freezing and drying. Leslie S, et al, “Trehalose lowers membrane phase transitions in dry yeast cells,” Biochim. Biophys. Acta, 1192:7-13 (1994); and Beattie G, et al, “Trehalose: a cryoprotectant that enhances recovery and preserves function of human pancreatic islets after long-term storage,” Diabetes 46:519-523 (1997). Also trehalose-based solutions form fragile glasses that protect proteins and cells. In low moisture environments, trehalose maintains thermodynamic stability of membranes by preserving phospholipid head group spacing and inhibiting lipid phase transitions and separation during freezing and drying. Glassy trehalose matrices slow down kinetic processes in stabilized samples by reducing water mobility and other relaxation processes.

Much of the knowledge in the field of biological preservation originates from work on protein and pharmaceutical stabilization by polymers and disaccharides. Hancock B & Zografi G, “Characteristics and significance of the amorphous state in pharmaceutical systems,” J. Pharm. Sci. 86:1-12 (1997): and Miller D, et al, “Stabilization of lactate dehydrogenase following freeze thawing and vacuum-drying in the presence of trehalose and borate,” Pharm. Res. 15:1215-1221 (1998). Polymers raise the glass transition temperature of the system, and disaccharides preserve protein structure during dehydration or freezing by forming glasses that alter interactions between protein and water. Miller et al., supra; and Sano F, et al., “A dual role for intracellular trehalose in the resistance of yeast cells to water stress,” Cryobiology 39:80-87 (1999). Recent X-ray crystallography data on lysozyme stabilized with trehalose suggests that trehalose does not directly interact with protein, but instead forms hydrogen bonds with water molecules surrounding protein, thereby altering the way in which water interacts with protein. Datta S, et al., “The effect of stabilizing additives on the structure and hydration of proteins: a study involving tetragonal lysozyme,” Acta Crystallogr. Biol. Crystallogr. 57:1614-1620 (2001).

In addition, trehalose exhibits a greater ability to hydrogen bond with water than other disaccharides. Ekdawi-Sever N, et al, “Molecular simulations of sucrose solutions near the glass transition temperature,” J. Phys. Chem. 105:734-742 (2001), thereby anticipating and confirming many of the findings mat are beginning to emerge from scattering experiments. The simulations of ionic species in disaccharide systems also allowed the creation of novel formulations containing cross-linked trehalose that have improved considerably the stability of cryopreserved and freeze-dried proteins. Miller D, et al., “Stabilization of lactate dehydrogenase following freeze thawing and vacuum-drying in the presence of trehalose and borate,” Pharm. Res. 15:1215-1221 (1998). These formulations are now being used commercially to stabilize a number of pharmaceutical and biological products, including PCR enzymes.

Cryopreservation and lyophilization of eukaryotic cells, such as hESCs, poses challenges that are not present with prokaryotic cells, such as bacteria. Whereas bacteria display stress responses to dehydration and to temperature extremes, eukaryotic cells do not. Likewise, bacteria possess a cell wall that imparts mechanical stability upon volume changes during freezing or drying, and may shield the cell membrane during ice crystal formation. In contrast, eukaryotic cells possess intracellular membranes that increase the number of structures requiring preservation and may provide additional barriers to protectant transport. Consequently, additional care must be taken during eukaryotic cell preservation to maintain cell integrity and cell viability.

Erogul et al. reported that intracellular trehalose concentrations on the order of 0.2 M allow approximately 75% of human keratinocytes or murine 3T3 fibroblasts to survive a freeze-thaw cycle that kills virtually all non-treated cells. Eroglu A, et al, “Intracellular trehalose improves the survival of cryopreserved mammalian cells,” Nat. Biotechnol. 18:163-167 (2000). In addition, Beattie et al. reported that the addition of trehalose formulations to cryopreserved human pancreatic islets doubled viable cell recovery and did not affect cell functions upon thawing. Beattie, et al., supra. Furthermore, Garcia de Castro & Tunnacliffe reported that trehalose concentrations of 80 mM increased the survival rate of a mouse fibrobiastoid cell line following partial dehydration induced by osmotic shock, but did not confer resistance to drying in air. Garcia de Castro A & Tunnacliffe A, “Intracellular trehalose improves osmotolerance but not desiccation tolerance in mammalian cells,” FEBS Lett. 487:199-202 (2000).

Preservation of pluripotent stem cells, especially hESCs, poses additional challenges. Not only must cells remain viable, but also must retain their differentiative capacity (i.e. remain pluripotent). Thus, certain signal transduction pathways must remain in place, and the stresses associated with freezing and drying must not induce premature or erroneous differentiation.

hESCs are extremely sensitive to the thermal and osmotic stresses experienced during cryopreservation. It remains unclear why hESCs are so sensitive; however, current hypotheses include differences in membrane compositions, fragile mitotic spindles, fracturing of cell-cell contacts, and slow rates of heat and mass transport through the multicellular colonies. Of the hESCs that do survive cryopreservation, a significant number differentiate shortly after thawing. As such, this premature or erroneous differentiation requires extra time and labor-intensive methods to isolate a pure hESC population.

New cryopreservation methods are sought to reduce cryopreservation-associated stresses.

### BRIEF SUMMARY

The present invention is summarized as providing methods and structures for stably freezing (cryopreserving) embryonic stem cells (ESCs), especially primate ESCs, including human ESCs (hESCs) adhered to, or maintained in, a microwell.

In a first aspect, a method for preparing an ESC colony for cryopreservation includes the steps of culturing embryonic stem cells on a first matrix portion in a microwell that supports growth of undifferentiated cells to form an ESC colony, providing on the cultured ESCs a second matrix portion that supports growth of undifferentiated cells to form a cryoprotection matrix-colony-matrix construct, exposing the structure to a cryoprotecting medium, optionally containing a carbohydrate, for a time sufficient to protect the viability of the cells in the matrix-colony-matrix construct, and then replacing the cryoprotecting medium with a freezing medium.

Advantageously, after freezing and thawing, colonies prepared in accord with the method maintain cell viability and can exhibit increased viability and decreased differentiation relative to cells cryopreserved in other ways, such as in suspension cultures. Viable cells are capable of normal growth and development after having been cryopreserved and thawed. Viability can be determined by a number of well-known methods, for example by MTT assay or Alamar Blue Assay. MTT (3-[4,5-dimethylthiazol-2-yl]-2,5-diphenyltetrazolium bromide) is a pale yellow substrate that is cleaved by living cells to yield a dark blue formazan product. The MTT assay is a safe, sensitive, in vitro assay for measuring cell proliferation and cell viability. The Alamar Blue Assay quantitatively measures proliferation of various human and animal cell lines, bacteria and fungi, detecting metabolic activity by incorporating a fluorometric/colorimetric growth indicator.

By “microwell,” we mean a bounded area having dimensions in the micrometer range. The bounded area is defined by a bottom and at least one side. The shape of the microwell can vary from circular (in which the at least one side is continuous) to any polygon (e.g., triangle, tetragon, pentagon, hexagon, etc.).

The cultured ESCs can be mammalian ESCs and can be primate or human ESCs and can be cryopreserved as colonies containing between about 1,000 and about 10,000 cells.

The colony can be formed on the first matrix portion in a microwell. The microwell can be rectangular, and can have a depth between about 50 μm and about 120 μm and lateral dimension between about 50 μm and about 600 μm on a side. Lateral microwell sides defined by a solid or semi-solid polymer matrix in which the wells are formed can be substantially the same length and can be between about 50 μm and about 100 μm on a side. In an array of microwells, the dimensions of the microwells can be varied as desired or can be consistent from one microwell to another. In some cases, volume per microwell can be consistent in the array, while length, width and/or depth can vary from microwell to microwell. Preferably, however, in an array, all microwells have consistent length, width and depth.

In some embodiments, the first and second matrix portions that support growth of undifferentiated cells can be layers beneath and above the cells, and in certain embodiments, the second matrix layer can be thinner than the first matrix layer. The second matrix portion maintains the attachment of the cells to the first matrix portion during the freezing process, with direct positive impact on viability of cells after thawing. Preferably, the first and the second matrix portions are porous. For example, the matrices can contain an extracellular matrix material, such as Matrigel® (BD Biosciences; a basement membrane preparation extracted from Engelbreth-Holm-Swarm (EHS) mouse sarcoma) in conditioned medium (CM), or other matrices that support growth of undifferentiated cells, such as a feeder layer of irradiated mouse embryonic fibroblasts (MEFs), especially as the first matrix. Use of MEFs can permit continuous undifferentiated growth and can obviate the need to use conditioned medium. Other alternatives can include but are not limited to collagen, hyaluronic acid, gelatin material, elastin, fibronectin (e.g., ProNectin®), laminin and mixtures thereof. The matrices can be porous or non-porous. A suitable, non-porous matrix that can be used to support cell growth is polystyrene coated with extracellular matrix (ECM) proteins or non-porous beads coated with ECM proteins.

The carbohydrate in the cryoprotecting medium can be, e.g., a disaccharide such as trehalose, provided in an amount and for a time effective to maintain viability after subsequent freezing and thawing. The cryoprotecting medium can be a conditioned medium. An exposure time effective to protect cell colonies of the type described herein can range from about 2 hours to about 30 hours, but shorter or longer times can be adequate to maintain a viability level acceptable for a particular use.

The freezing medium can be a conventional ESC freezing medium. The freezing medium can include, e.g., between about 5% and about 15% by volume of DMSO and serum (e.g., FBS) concentrations can range from 20% to 95% in human embryonic stem cell (HES) medium, for example, 10% DMSO, 30% FBS and 60% conditioned HES medium.

The matrix-colony-matrix construct prepared in accord with the method can be cooled to and stored at conventional cryopreservation temperatures, such as at a temperature ranging from about −70° C. to about −195° C.

In a second aspect, the invention is summarized in that the matrix-colony-matrix construct prepared in the method for cryopreservation includes a cultured ESC colony provided between first and second matrices. Advantageously, the structure is provided in a microwell having properties described above in connection with the related method. In some embodiments, the structure contains a cryoprotecting medium or a freezing medium.

In a third aspect, a method for cryopreserving an ESC colony for cryopreservation includes the steps of culturing embryonic stem cells on a first matrix portion in a microwell that supports growth of undifferentiated cells to form an ESC colony, providing on the cultured ESCs a second matrix portion that supports growth of undifferentiated cells to form a cryoprotection matrix-colony-matrix construct, exposing the structure to a carbohydrate-containing cryoprotecting medium for a time sufficient to protect the viability of the cells in the matrix-colony-matrix construct, replacing the cryoprotecting medium with a freezing medium, and freezing the construct.

These and other features, aspects and advantages of the present invention will become better understood from the description that follows. In the description, reference is made to the accompanying drawings, which form a part hereof and in which there is shown by way of illustration, not limitation, embodiments of the present invention. The description of preferred embodiments is not intended to limit the present invention to cover all modifications, equivalents and alternatives. Reference should therefore be made to the claims recited herein for interpreting the scope of the present invention.

### BRIEF DESCRIPTION OF THE DRAWINGS

The present invention will be better understood and features, aspects and advantages other than those set forth above will become apparent when consideration is given to the following Description which refers to the following drawing, wherein:

FIG. 1 depicts the manufacture of polymeric substrates containing microwells and shows resulting substrates and microwells treated as described to produce hESC cultures of defined size, shape and volume.

FIGS. 2A-B depict two separate samples of hESCs (H1, passage 44) cultured in microwells that were frozen directly in a plate. After thawing, the cells were continuously grown in the microwells for six days. Then, the cells were washed once with 1× PBS and re-suspended in 2 ml of 1× PBS per well. 2 μl of Calcein AM was added to the cells, and the plate was incubated at 37° C. for 30 minutes. Fluorescence images were taken at 10× magnification. Cells surviving the freeze-thawing process were stained green and kept in the wells.

FIG. 3 depicts improved survival rates of hESCs from microwells when compared to standard techniques (i.e. TCPS). HESCs were frozen for four weeks in microwells and analyzed four hour post-thaw for metabolic activity using a Calcein AM reduction assay. Y-axis: % cells metabolieally active. X-axis: cell sample type, including fresh hESCs, control hESCs (TCPS), hESCs cryopreserved in microwells of 50×100 μm and hESCs cryopreserved in microwells of 50×200 μm, with a depth of ˜50 μm.

### DESCRIPTION OF PREFERRED EMBODIMENTS

The present invention relates to the observation that culturing hESCs in a microwell environment improves cell viability and maintains hESC pluripotency. This observation suggests that hESC viability and pluripotency can be maintained in a microwell during cryopreservation.

A method for preparing at least one ESC colony for cryopreservation is described. The method can be conveniently practiced in a microwell suited for culturing ESC colonies. Upper and lower faces of the colony prepared in the method are protected by first and second solid porous matrices that define the matrix-colony-matrix construct. If cultured in microwells, lateral faces of the colonies are further protected by the microwell walls. In the method, the matrix-colony-matrix construct is exposed to a cryoprotecting medium that optionally includes a carbohydrate and then to a freezing medium. After the method is complete, the prepared colony can be frozen and then thawed. Thawed cells prepared in accord with the method exhibit maintained or increased cell viability (at least about 80%, or at least about 90%, or at least about 95% of the thawed cells are viable after thawing) and decreased cell differentiation relative to colonies prepared for cryopreservation in other ways.

It is desirable to prepare the colonies for cryopreservation in a microwell. U.S. patent application Ser. No. 11/765,831, filed Jun. 20, 2007, incorporated herein by-reference as if set forth in its entirety, describes formation of molded, recessed microwells in a polymer matrix on a solid substrate, preparation of the microwells for cell culture, and ESC culture conditions. Briefly, portions of the microwells at and near the well bottoms can be made attractive to cellular adhesion using extracellular matrix materials, while upper portions, and portions of the polymer matrix outside of the micro well(s) can be made resistant to cell adhesion using protein-resistant self assembling monolayers (SAM). Advantageously, a plurality of microwells can share uniform length, width and depth dimensions such that the colony in each microwell is characterized by a consistent well-to-well volume, cell number and colony shape. In the microwells, the ESCs remain substantially undifferentiated (i.e., between 90% and 95% of the cells remain undifferentiated) for at least about three weeks when grown in a non-differentiating medium. The substantially undifferentiated cells retain the ability to self-renew and can be plated and passaged like hESCs in conventional culture.

Unless defined otherwise, all technical and scientific terms used herein have the same meaning as commonly understood by one of ordinary skill in the art to which the invention belongs. Although any methods and materials similar to or equivalent to those described herein can be used in the practice or testing of the present invention, the preferred methods and materials are described herein.

The invention will be more fully understood upon consideration of the following non-limiting Examples.

### EXAMPLES

### Example 1

### 3-D Microwells for Culturing Embryonic Stem Cells

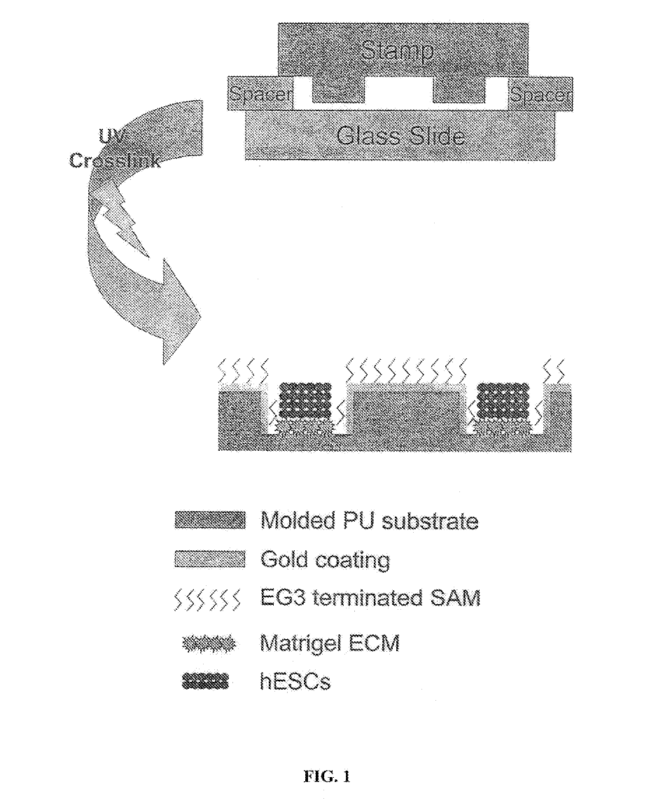

Reference is made to FIG. 1. Microscope slides having a homogeneous distribution of wells of identical size and shape were constructed in three steps using a polydimethylsiloxane (PDMS) stamp to shape a surface of a UV-crosslinkable polyurethane polymer matrix.

First, silicon masters each having desired microwell patterns formed into a surface thereof were prepared using photolithography and plasma etching techniques similar to those used by Chen et al. Chen C, et al., “Using self-assembled monolayers to pattern ECM proteins and cells on substrates,” Methods Mol. Biol. 139:209-219 (2000), incorporated herein by reference as if set forth in its entirety. The surfaces were passivated by fluorination with (tridecafluoro-1,1,2,2,-tetrahydrooctyl)-1-trichlorosilane vapor. Second, a mixture of PDMS elastomer prepolymer with curing agent (10:1) (Sylgard 184 Silicon Elastomer; Dow Corning; Midland, Mich.) was poured over the silicon masters to form PDMS stamps. The mixture was degassed under vacuum and incubated overnight at 70° C. to promote polymerization. Finally, PDMS stamps were clipped on two sides to glass microscope slides separated by 250 μm spacers. Norland optical adhesive 61 (Norland Products Inc.; Cranbury, N.J.) prepolymer was fed to one end of the clipped stamps and distributed via capillary action. After crosslinking under UV light for two hours, stamps and spacers were removed, yielding patterned microwells on the slides. Using these techniques, microwells were created with lateral dimensions of about 50 μm×50 μm to about 600 μm×600 μm, and with depths from about 50 μm to about 120 μm.

The surfaces of the slides, but not the microwells themselves, were coated with gold by e-beam evaporation using oblique angles to restrict gold evaporation to the inter-well portions of the surface and to the sides of the microwells. Two evaporations were performed, with slides rotated 90° between evaporations. A 20 Å titanium layer preceded a 200 Å gold layer evaporation. The resulting gold-treated array of microwells was semi-transparent, allowing use of light microscopy during culture. The microwells were washed in 100% ethanol and sterilized under UV light for one hour. Slides were placed in individual wells of a 6-well culture dish with 2 ml/well of a 2 mM tri-ethylene glycol-terminated (Prochimia; Sopot, Poland) alkanethiol ethanoic self-assembling monolayer (SAM) solution. Slides were incubated at room temperature for 2 hours and washed in 100% ethanol. All SAM solutions were stored at 4° C. and used within one week.

A solution of cell-attracting Matrigel® (BD Biosciences; Franklin Lakes, N.J.) was then provided inside the microwells, where gold was not deposited. Matrigel®-coated microwells were washed once in PBS and were then transferred to 6-well polystyrene plates. Non-tissue-culture-treated plates were used, to prevent cells from attaching to the plate surface around the microwell slides. hESCs (H1 or H9, passage 20-45; WiCell; Madison, Wis.) from wells of a 6-well plate at normal passaging confluency were treated with 1 ml/well trypsin (Invitrogen; Carlsbad, Calif.) pre-warmed to 37° C. To prevent hESC colonies from dissociating into single cells, plates were monitored under a microscope and when hESCs at colony edges began to dissociate, trypsin was neutralized with 2 ml/well mouse embryonic fibroblast (MEF)-conditioned medium. hESCs were gently washed from the plate and pelleted. The pellet was re-suspended in 0.75 ml/sample MEF-conditioned medium supplemented with 4 ng/ml bFGF (CM/F⁺).

hESCs were then seeded in aliquots onto 1 to 2 microwells having 50 μm or 100 μm lateral dimensions, taking care to retain the entire cell solution on top of the slides. Samples were incubated for 30 minutes at 37° C. to allow hESCs to settle into the microwells before adding 1.5 ml/well CM/F⁺. The medium was changed daily thereafter and the cells typically reached confluence within a week.

Using phase contrast microscopy to visualize hESCs, as well as Hoechst DNA-binding dye staining, it was determined that hESCs localized only to the insides of the wells. The desired hESC localization was obtained in microwells having lateral dimensions ranging from 50 μm/side to 600 μm/side. After several days in culture, bubbles appeared in the substrate; however, microwell integrity remained intact.

Phase contrast and epifluorescence images of differentiation data were obtained on an Olympus IX70 model microscope (Leeds Precision Instruments; Minneapolis, Minn.) using MetaVue 5.0rl imaging software. Phase contrast, brightfield and epifluorescence images of hESC localization and viability were obtained on a Leica DM ARB microscope (Leica Microsystems, Inc.; Bannockburn, Ill.).

### Example 2

### Cryopreservation of Embryonic Stem Cells

To prepare a Matrigel® plate, a tube of Matrigel® stock (2 mg) was taken directly from storage at −20° C. A Matrigel® pellet was immediately re-suspended in 6 ml ice-cold DMEM/F12. All chunks in the mixture were eliminated by vigorous pipetting. A 1 ml aliquot of the mixture was added to each well of a 6-well plate. The plate was maintained at room temperature for 1 hour or overnight at 4° C. before use.

To prepare conditioned medium, a flask was coated with 0.1% gelatin solution, 10 ml to a T75 flask. After the flask was coated, it was incubated overnight in a 37° C., humidified incubator with 5% CO₂ for 24 hours prior to plating irradiated MEF cells. 15 ml of irradiated MEF cells a concentration of 2.12×10⁵ cells/ml MEF medium (90% DMEM, 10% FBS and 1% MEM non-essential amino acids solution) were added to a T75 flask and incubated overnight. The MEF medium was aspirated away and 20 ml HES medium without bFGF (80% DMEM/F12 medium, 20% Knockout Serum Replacement, 1% L-glutamine solution and 0.1 mM MEM non-essential amino acids solution) was added to the flask. The flask was again incubated overnight. The medium was collected and 20 ml of fresh HES medium without bFGF was added to the flask. Every day for up to two weeks, the medium was collected. Then, bFGF was added to the collected medium to a final concentration of 4 ng/ml before use with hESCs.

hESCs were grown to approximately 1,000 to 10,000 cell colonies on Matrigel® in conditioned medium or on MEF feeder cells. A thin top (0.1 mM to 1 mM) layer of Matrigel® (6 mg for one 24-well plate diluted in 12 ml CM/F⁺, 0.5 ml/well) was poured over the cell colonies, effectively creating a matrix-colony-matrix construct. The plates were incubated at 37° C. for 1 hour. Excess Matrigel® solution, but not the construct, was aspirated off and replaced with 0.5 ml/well 35 mM trehalose in conditioned HES medium.

The plates were incubated for one day. The cryoprotecting medium on the plate was aspirated off and replaced with 0.5 ml fresh freezing medium (10% FBS, 30% DMSO and 60% conditioned-HES medium) made on the day of freezing. The edge of the plate was sealed with Parafilm®. The plate was wrapped with one layer of Saran® wrap and with five layers of paper towels, then was put into a styrofoam box and placed into a freezer until frozen at −80° C. The freezing rate using this insulation method was about 1° C./minute. The box was then stored in liquid nitrogen.

Before thawing, the plates were taken out of the box and the paper towels were removed. The plates were placed in a 37° C. water bath and thawed as rapidly as possible. After thawing, 1 ml of fresh conditioned HES medium was added dropwise to each well. The medium was carefully aspirated away and replaced with fresh HES medium (0.5 ml). The plates were incubated at 37° C. Medium was changed daily and cells were passaged when colony size was greater than about 10,000 cells.

To measure the viability of cryopreserved hESCs, an MTT assay was conducted. Briefly, hESCs were grown on a fiat-bottomed 24-well tissue culture plate, with 0.5 ml growth medium in each well. MTT solution (0.05 ml) was added to each well and mixed by tapping gently on the side of the plate tray. The plate was incubated at 37° C. for 2 to 4 hours to permit MTT cleavage. Isopropanol (0.5 ml) with 0.04 N HCl was added to each well and again mixed thoroughly by repeated pipeting. Absorbance was measured on an ELISA plate reader within an hour at a wavelength of 595 nm.

In addition, viability of cryopreserved hESC was measured using an Alamar Blue Assay. Briefly, hESCs were grown on a flat-bottomed, 24-well tissue culture plate, with 0.5 ml growth medium in each well. Alamar Blue solution (0.05 ml) was added to each well and mixed by tapping gently on the side of the plate tray. The plate was incubated at 37° C. for 3 hours. Absorbance was measured on an ELISA plate reader at a wavelength of 595 nm. Typical results using the matrix-colony-matrix sandwich method for cryopreservation of hESCs showed improved survival rate (i.e., cell viability) from 0.1% to 1%, which is typically observed with current cryopreservation methods, up to 10%.

### Example 3

### Cryopreservation of Encapsulated hESCs in 3-D Microwells

3-D microwells were created as described in Example 1 and treated in accord with the method of Example 2. Microwells were treated with Matrigel®, which selectively absorbs to the bottom of the wells. hESCs were seeded at 1-5 ×10⁻⁵ cells/micro well and allowed to grow until they filled the microwells. Culture conditions were as described above. Although CM/F⁺ was changed daily, the cells were not passaged. Prior to freezing, the hESCs were treated as described above in Example 2 to form matrix-colony-matrix constructs (i.e., the microwells were covered with a top layer of Matrigel® and treated with a carbohydrate-based cryopreservation medium, followed by a freezing medium). The hESCs were frozen and stored at −80° C. or in liquid nitrogen.

hESCs were thawed and then cultured in the microwells or harvested by dispase treatment and either transferred to new microwells or to MEF monolayers or Matrigel®-coated plates. Virtually all colonies frozen in microwells were recovered after 2 to 4 weeks at −80° C. After 5 days of recovery, over 80% of the cells in the culture are viable and undifferentiated.

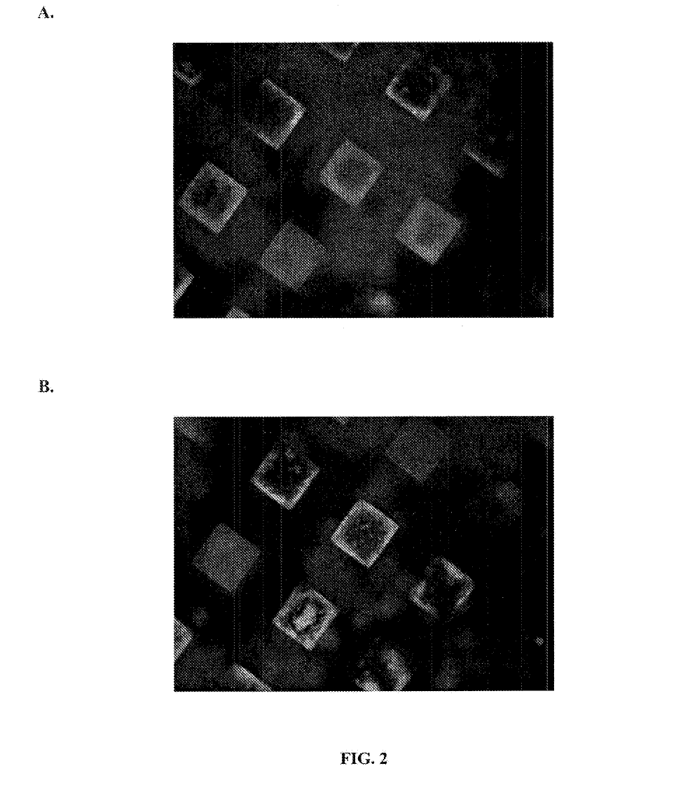

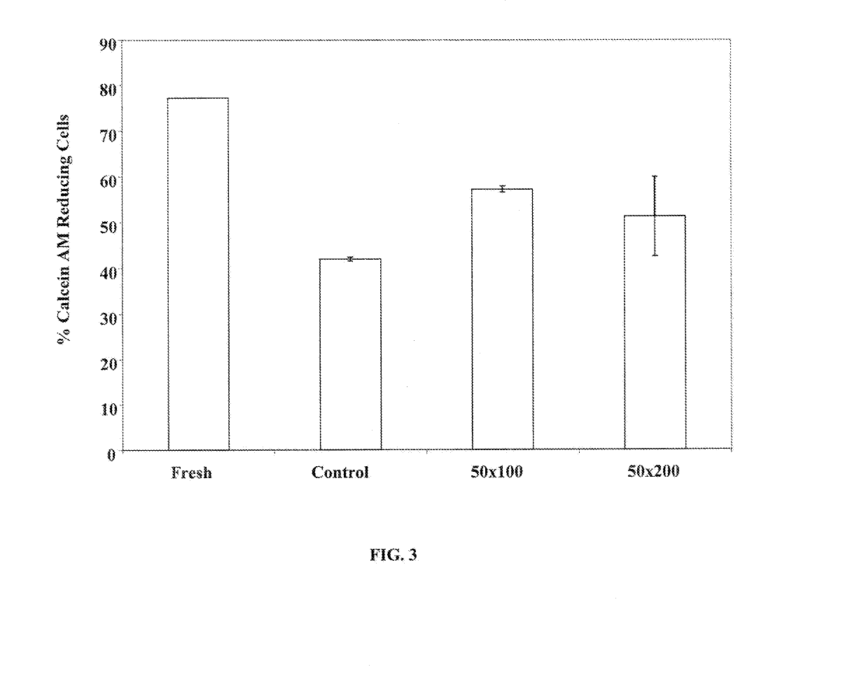

Viability of cryopreserved hESC was measured using a Calcein AM Reduction Assay according to the manufacture's instruction. Briefly, hESCs were frozen for four weeks in microwells and analyzed four hours post-thaw for metabolic activity. Cells in 50×100 microwell, 50×200 microwells and TCPS controls yielded 57%, 51% and 42%, viability, respectively; whereas fresh hESC cultures yielded 77% viability (FIGS. 2-3).

The present invention has been described in connection with what are presently considered to be the most practical and preferred embodiments. However, the present invention has been presented by way of illustration and is not intended to be limited to the disclosed embodiments. Accordingly, those skilled in the art will realize that the present invention is intended to encompass all modifications and alternative arrangements within the spirit and scope of the present invention as set forth in the appended claims.

The invention claimed is:

1. A method of preparing embryonic stem cells for cryopreservation, the method comprising the steps of:
culturing embryonic stem cells on a first matrix in a microwell that supports growth of undifferentiated embryonic stem cells to form an embryonic stem cell colony;
providing on the embryonic stern cell colony a second matrix that supports growth of undifferentiated embryonic stem cells to form a cryoprotective matrix-colony-matrix construct;
exposing the construct to a cryoprotecting medium for a time sufficient to protect the viability of the cells in the matrix-colony-matrix construct; and
replacing the cryoprotecting medium with a freezing medium.

2. The method of claim 1, wherein the cells are mammalian cells.

3. The method of claim 1, wherein the cells are primate cells.

4. The method of claim 1, wherein the cells are human cells.

5. The method of claim 1, wherein the colonies comprise between about 1,000 and about 10,000 cells.

6. The method of claim 1, wherein at least one of the first and second matrices comprises an extracellular matrix material and conditioned medium.

7. The method of claim 1, wherein the extracellular matrix material is selected from the group consisting of a basement membrane preparation, collagen, hyaluronic acid, gelatin, elastin, fibronectin, laminin and mixtures thereof.

8. The method of claim 1, wherein the second matrix portion is thinner than the first matrix portion.

9. The method of claim 1, wherein the cyroprotecing medium comprises a carbohydrate.

10. The method of claim 9, wherein the carbohydrate is a disaccharide.

11. The method of claim 10, wherein the carbohydrate is trehalose.

12. The method of claim 1, wherein the freezing medium comprises fetal bovine serum (FBS), dimethyl sulfoxide (DMSO) and conditioned human embryonic stem cell (HES) medium.

13. The method of claim 1, wherein the cryoprotecting medium is exposed to the matrix-colony-matrix construct for between about two and about thirty hours.

14. The method of claim 1, wherein the microwell comprises a depth between about 50 μm and about 120 μm and lateral dimension between about 50 μm and about 600 μm on a side.

15. The method of claim 1, wherein the microwell is rectangular.

16. A method of cryopreserving embryonic stem cells the method comprising the steps of:
culturing embryonic stem cells on a first matrix in a microwell that supports growth of undifferentiated embryonic stem cells to form an embryonic stem cell colony;
providing on the cultured embryonic stem cells a second matrix that supports growth of undifferentiated embryonic stem cells to form a cryoprotective matrix-colony-matrix construct;
exposing the construct to a cryoprotecting medium comprising a carbohydrate for a time sufficient to protect the viability of the cells in the matrix-colony-matrix construct;
replacing the cryoprotecting medium with a freezing medium; and
freezing the construct.

17. A cryopreservation construct comprising first and second matrix portions and embryonic stem cells therebetween.

18. The cryopreservation construct of claim 17, further comprising a polymer matrix defining at least one microwell having a bottom and lateral sides, the construct being provided in the microwell.

19. The cryopreservation construct of claim 18, the first matrix portion being associated with the bottom of the at least one microwell.

20. The construct of claim 18, the at least one microwell having a depth between about 50 μm and about 120 μm and a lateral side dimension between about 50 μm and about 600 μm.

21. The matrix-colony-matrix construct of claim 17, wherein the lateral sides are of equal dimension.
